(19) United States
(12) Patent Application Publication
(10) Pub. No.: US 2006/0035355 A1
(43) Pub. Date: Feb. 16, 2006

(54) Method for producing sugar and a useful material

(75) Inventors: Satoshi Ohara, Moriya-shi (JP); Yoshitaka Tomino, Moriya-shi (JP); Akira Sugimoto, Nishino-omote-shi (JP); Kunihiro Ujihara, Nishino-omote-shi (JP); Yoshifumi Terajima, Nishino-omote-shi (JP)

Correspondence Address: HOFFMANN & BARON, LLP, 6900 JERICHO TURNPIKE, SYOSSET, NY, 11791, US

(73) Assignee: ASAHI BREWERIES, LTD.

(21) Appl. No.: 11/244,581

(22) Filed: Oct. 6, 2005

Related U.S. Application Data
(63) Continuation of application No. PCT/JP04/04962, filed on Apr. 6, 2004.

(30) Foreign Application Priority Data
Apr. 7, 2003 (JP) 2003-102534
Feb. 3, 2004 (JP) 2004-027016

(51) Int. Cl. C12P 7/06; C13J 1/06

(52) U.S. Cl. 435/161; 127/46.1

(57) ABSTRACT

An object of the present invention is to provide a method for producing sugar and ethanol from sugar cane, in which almost all of energy to be consumed in the production processes of the sugar, the ethanol and the like can be supplied by the energy obtained by burning a pressed residue of sugar cane, yet without decreasing the sugar amount to be produced. The present invention provides a method for producing sugar and a useful material from sugar cane, comprising the steps of: (a) producing from sugar cane a pressed juice and pressed residue of sugar cane; (b) producing sugar and blackstrap molasses from said pressed juice; and (c) generating an energy and a useful material by using said pressed juice, said blackstrap molasses and said pressed residue of sugar cane as source materials that have been obtained from said steps (a) and (b), wherein said sugar cane contains an amount of 15% or greater by mass of fiber component in its cane stem region and provides a dry matter yield amount per unit area of 40 t/ha/year or higher; and 90% or more of energy required for all of the steps of said production method is obtained from energy generated by burning said pressed residue of sugar cane.

### CROSS-REFERENCE TO RELATED APPLICATION

This application is a continuation of International Application No. PCT/JP2004/004962, which designates the U.S., filed Apr. 6, 2004, which claims priority to Japanese Application No. 2003-102534, filed Apr. 7, 2003 and Japanese Application No. 2004-027106, filed Feb. 3, 2004, the contents all of which are incorporated by reference herein.

### BACKGROUND OF THE INVENTION

The present invention relates to a method for producing sugar and alcohol, plastic and the like from sugar cane by using blackstrap molasses as a source material derived therefrom.

Ethanol derived from the vegetable to be used as a fuel has been expected to be a liquid fuel substituting for a gasoline in order to prevent an increase of carbon dioxide gas. Regarding a method for producing the ethanol derived from the vegetable, one method using sugar cane as a source material is conventionally known (see FIG. 1). Advantageously, in this method, almost all of the energy required for producing the ethanol can be obtained from the energy generated by burning the sugar cane residue from its squeezing or pressing process (hereafter referred to as pressed residue of sugar cane). However, there is a problem in association with the case of using the sugar cane as a source material for the ethanol that due to a competition with the sugar production, any production of the ethanol from the sugar cane supplied from the existing area under cultivation may lead to a decrease in sugar production volume available as foodstuff.

Generally, in producing of sugar (raw sugar) from sugar cane, a method illustrated in FIG. 2 is employed to thus produce the sugar. In this regard, such a method for producing the ethanol has been also suggested that uses blackstrap molasses which is a by-product from the producing process of the sugar (see FIG. 3). According to this method, sugar cane having a fiber component content in a range of 10 to 20% by mass is typically used and while producing the sugar, a pressed residue of sugar cane is burnt so as to supplement the energy required for producing the ethanol. Thus, although it can solve the aforementioned problem of decrease in the sugar production, the energy obtainable by burning the pressed residue of sugar cane will be too small to supply all the energy to be consumed in the sugar producing process, which may call for a situation that the shortage of energy has to be compensated for by the energy obtainable from an electric power source or a heavy oil. Further disadvantageously, because of a small amount of blackstrap molasses, the above method yields only a small amount of ethanol to be obtained.

### SUMMARY OF THE INVENTION

Accordingly, an object of the present invention is to provide a method for producing sugar and ethanol from sugar cane, which can increase a production amount of ethanol without decreasing that of sugar, said method characterized in that almost of all the energy to be consumed in the production processes of the sugar and the ethanol can be supplied by the energy obtained by burning a pressed residue of sugar cane that is to be resultantly discharged in the production processes of the sugar and the ethanol from the sugar cane.

To address the above-pointed problem, the inventors of the present invention have devoted themselves in an enthusiastic research and ultimately found that optimizing the production method by using such sugar cane that contains 15% or greater by mass of fiber component particularly in its cane stem region can provide the sugar production and the ethanol production in a compatible manner as well as in energy efficient manner. Based on this finding, the present invention has been made.

That is, the present invention provides a method for producing sugar and a useful material from sugar cane, comprising the steps of:
(a) producing from sugar cane a pressed juice and pressed residue of sugar cane;
(b) producing sugar and blackstrap molasses from said pressed juice; and
(c) generating an energy and a useful material by using said pressed juice, said blackstrap molasses and said pressed residue of sugar cane as source materials that have been obtained from said steps (a) and (b), wherein
said sugar cane contains an amount of 15% or greater by mass of fiber component in its cane stem region and provides a dry matter yield amount per unit area of 40 t/ha/year or higher; and
90% or more of energy required for all of the steps of said production method is obtained from energy generated by burning said pressed residue of sugar cane.

According to the production method of the present invention, almost of all the energy required in all of the production processes of the present invention can be obtained from the energy generated by burning the pressed residue of sugar cane.

Further, the present invention enables a useful material, for example, ethanol to be produced without leading to the decrease in the production amount of the sugar.

Since a single system can be used to produce the sugar and the ethanol from the sugar cane, or the source material, the sugar and the ethanol can be produced in an energy efficient manner.

Since a number of crystallizing process to be required for producing the sugar can be reduced, the generation of chemical product from Maillard reaction can be suppressed, consequently preventing the coloring and the generation of fermentation inhibitor (such as furfural). Yet further, since the number of crystallizing process of the sugar can be reduced, a concentration of salinity to sugar which has been conventionally considered problematic in the application of the blackstrap molasses as a fermentation source material (i.e. the problem pointed in the Japanese Patent Laid-open Publication No. Sho 7-59187) may been reduced, and thus it will become possible to employ even such a fermentable microorganism having no salt tolerance.

### BRIEF DESCRIPTION OF THE DRAWINGS

FIG. 1 schematically illustrates a method for producing ethanol from sugar cane;

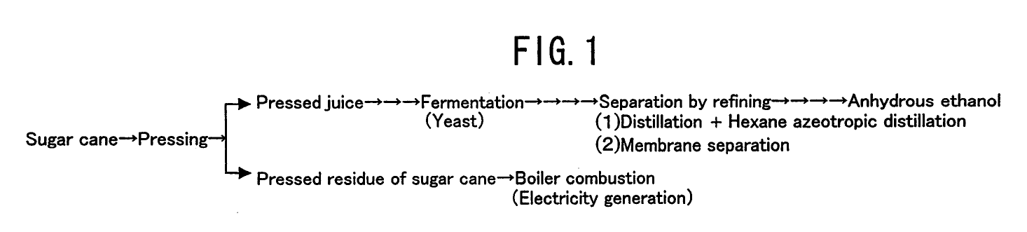

FIG. 2 schematically illustrates a method for producing sugar from sugar cane;

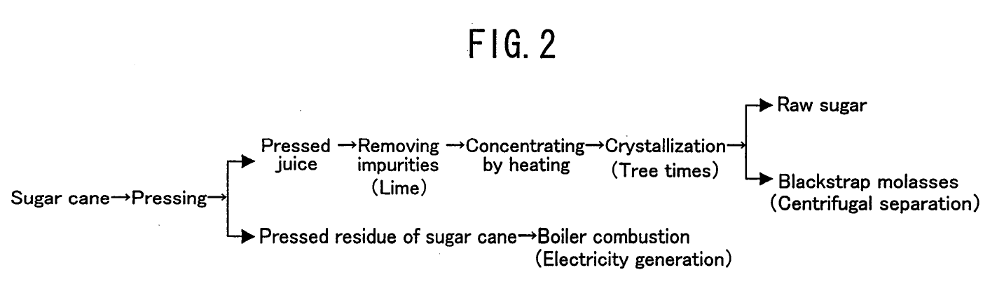

FIG. 3 schematically illustrates a method for producing ethanol from blackstrap molasses;

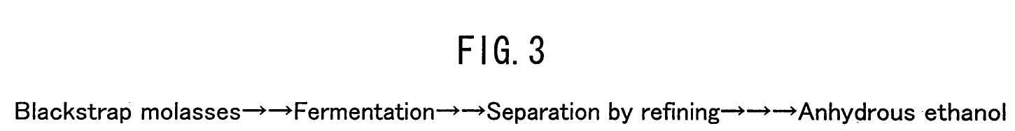

FIG. 4 shows an exemplary production flow in a production method of raw sugar and blackstrap molasses according to the present invention;

FIG. 5 shows a mass balance in the crystallization of sugar;

FIG. 6 is a schematic diagram of an example of a production method of ethanol;

FIG. 7 shows an exemplary production flow in a production method of ethanol;

FIG. 8 is a schematic diagram of an example of a production method of sugar and ethanol according to the present invention;

FIG. 9 shows a calculation method of a combustion energy of bagasse;

FIG. 10 is a graphical representation of a relationship among a number of crystallizing processes, a raw sugar yield amount and a raw sugar yield ratio;

FIG. 11 is a graphical representation of a cane sugar residual ratio in molasses; and

FIG. 12 is a graphical representation of HMF and chromaticity in molasses.

### DESCRIPTION OF THE PREFERRED EMBODIMENTS

A method for producing sugar and ethanol according to the present invention comprises the steps of:
(a) producing from sugar cane a pressed juice and a pressed residue of sugar cane;
(b) producing sugar and blackstrap molasses from said pressed juice; and
(c) generating an energy and a useful material by using said pressed juice, said blackstrap molasses and said pressed residue of sugar cane as source materials that have been obtained from said steps (a) and (b).

A process for preparing the pressed juice and the pressed residue of sugar cane from the sugar cane may be carried out in any method known to those skilled in the art, including a pressing process, for example. Specifically, a cane stem portion of reaped sugar cane is cut into 15 to 30 cm long pieces by a cutter, which are then shredded finely by a shredder and processed by a mill roll to press out the juice. In order to enhance the pressing-out rate, water is poured to an end roll so as to press out 95 to 97% of sugar content. Subsequently, the resultant product is heated up to 80 to 100% by a juice heater and treated in a lime mixing bath, where it is added with the lime to allow any impurities to be precipitated as a lime salt and a supernatant liquid to be concentrated by evaporation. A resultantly obtained pressed juice primarily includes sucrose, glucose and so on. The pressed residue of sugar cane primarily includes cellulose, hemicellulose, lignin and so on.

For the purpose of the specification of the present invention, the term “sugar cane” represents a perennial herb typically categorized to Gramineae, Panicoideae, Andropogoneae or Saccharum L., including six kinds thereof, Saccaharum spontaneum L., Saccharaum officinarum L., Saccharum robustum Jeswiet, Saccharum Barberi Jeswiet, Saccharum sinense Roxb., and Saccharum edule as well as any interspecific hybrids among them, and further including any intergeneric hybrids with vegetables of related genus (i.e., the Miscanthus genus, the Sorghum genus, the Erianthus genus, the Ripidium genus and so on), which contain an amount of 5% or more of sugar to be produced (i.e., sucrose). It is to be noted that the interspecific hybrids and the intergeneric hybrids are collectively referred to as the Saccharum hybrids. The sugar cane used in the production method of the present invention is represented by any one selected from a group consisting of the hybrids by the interspecific cross among the vegetables of the Saccharum L., the hybrids by the intergeneric cross between the vegetables of the Saccharum L. and of the related genus (i.e., the Sorghum genus, the Miscanthus genus, the Erianthus genus, the Ripidium genus and so on), and the hybrids created by crossing among said three different genus, which is further defined by a fiber component content of 15% by mass or greater, preferably in a range of 20 to 25% by mass, in a cane stem portion of the sugar cane, as cultivated for one year in accordance with a typical ratooning cultivation for the sugar cane in a field in a temperate zone. For the case with the fiber component content of 15% by mass or greater, 90% or more of the energy to be required in all of the processes in the production method of the present invention can be obtained from the amount of energy generated by burning the pressed residue of sugar cane as described above. Preferably, 95% or more, and most preferably, 100% of the energy to be required in all of the processes in the production method of the present invention can be obtained.

In this regard, the measurement of the fiber component content in the cane stem portion of the sugar cane may be performed in accordance with the method defined in the Sugar Manufacturing Chemical Handbook (published by Japanese Molasses Industrial Association). To illustrate one exemplary method, the measurement of the fiber component content may be performed in accordance with the following procedure.

(1) A set of 10 pieces of cane stem of the sugar cane (samples to be used in measurement) is shredded finely by a shredder.

(2) A sample portion of 500 g is measured out of said shredded samples.

(3) Said sample portion of 500 g is pressed by a hydraulic presser.

(4) A mass of residue (a juice pressed-out bagasse weight) is measured, and the residue is put into a cloth bag and dried by a dryer.

(5) After drying at 90% for 48 hours or longer, the bagasse mass after drying (a dried bagasse weight) is measured.

(6) Bagasse fiber weight is calculated from the following equation:
Bagasse fiber weight=Dried bagasse weight−(Juice pressed-out bagasse weight−Dried bagasse weight)×Pressed juice/(100−Pressed juice).

(7) Subsequently, a fiber component content is calculated from the following equation: Fiber component content=Bagasse fiber weight/500 g×100.

The sugar cane used in the production method of the present invention is represented by one of high-yielding variety defined by a dry matter yield amount of 40 t/ha/year or more. With such a yield amount, there would be no decrease in the amount of sugar to be produced. Further, in order to efficiently produce the sugar and the useful material, especially alcohol and/or plastic, the dry matter yield amount per unit area should be preferably 65 t/ha/year or greater, more preferably 80 t/ha/year or greater. The dry matter yield amount of the sugar cane per unit area may be measured by, for example, the following procedure.

(1) A sample set of 5 pieces of moderate growing sugar cane stem is selected from reaped sugar cane stems (sampling should be made carefully so as not to remove the dead leaves, as much as possible).

(2) Every one of selected 5 pieces of cane stem has its raw weight measured with its dead leaves and/or the head portion left as they were.

(3) The sample set of 5 cane stems that has its raw weight measured is packed in a net and dried in a dryer (the drying time may be varied depending on the actual condition of the cane stems, and the drying of the cane stems may typically take longer time than the pressed residue of sugar cane because the stems are difficult to dry).

(4) After having been dried, a dry mass is measured for the 5 pieces of cane stem.

(5) A dry matter ratio is calculated from the following equation:
Dry matter ratio=Dry mass of 5 pieces of cane stem/Raw weight for 5 pieces of cane stem×100

(6) The total raw weight for the entire yield per unit area (including dead leaves and top head portions) is multiplied by the determined dry matter ratio so as to obtain the dry matter amount per unit area.

Said sugar cane used in the production method of the present invention may include, for example, such sugar canes that have been bred and developed by the inventors of the present invention, including: 95GA-27, S8-42, KRSp93-21 and KRSp93-30 (Akira Sugimoto, Tropical Zone Agriculture, 46, Extra Issue 2, p49-50 (2002)); S3-32, S3-10, SY480, SY435, SY478 and 97S-133 (Akira Sugimoto, Tropical Zone Agriculture, 45, Extra Issue 2, p57-58 (2001)); and S3-31 (Akira Sugimoto, Tropical Zone Agriculture, 45, Extra Issue 2, p59-60 (2001)). The fiber component content and dry matter yield amount for those varieties of sugar cane are indicated in Table 1. It is to be noted that Table 1 also indicates the averaged value over the common varieties of sugar cane along with the data regarding to the conventional variety (NCo310).

TABLE 1

| | Dry matter yield | Fiber component |
|---|---|---|
| Variety of sugar cane | amount (t/ha/year) | content (% by mass) |
| 95GA-27¹⁾ | 66.4 | 20.5 |
| S3-32²⁾ | 91.4 | 20.8 |
| S3-10²⁾ | 90.8 | 23 |
| SY480²⁾ | 84.7 | 21.1 |
| SY435²⁾ | 72 | 17.1 |
| SY478²⁾ | 71.7 | 17.3 |
| 97S-133³⁾ | | 19.1 |
| S3-31⁴⁾ | 73 | 15.7 |
| S8-42¹⁾ | 44.6 | 19.1 |
| KRSp93-21¹⁾ | 53.1 | 22.4 |
| KRSp93-30¹⁾ | 58.5 | 22.5 |
| Common varieties of | 17.0 | 12.0 |
| sugar cane (Averaged | | |
| value over conventional | | |
| varieties: NCo310, | | |
| NiF5, NiF8, Ni12)²⁾ | | |
| NCo310 (Conventional | 14.2 | 10.5 |
| variety)⁴⁾ | | |

Remarks:
¹⁾3-times repetitive randomized block method (Akira Sugimoto, Tropical Zone Agriculture, 46, Extra Issue 2, p49-50 (2002));
²⁾The cultivation period for about 9 months, no repetition (Akira Sugimoto, Tropical Zone Agriculture, 45, Extra Issue 2, p57-58 (2001));
³⁾The cultivation period for 12 months, no repetition (Akira Sugimoto, Tropical Zone Agriculture, 45, Extra Issue 2, p57-58 (2001)); and
⁴⁾The cultivation period for about 150 days, no repetition (Akira Sugimoto, Tropical Zone Agriculture, 45, Extra Issue 2, p59-60 (2001)).

Conventionally, the recommended varieties of sugar cane suitable for the sugar production have been represented by those having a high cane sugar (sucrose) content but a low fiber component content. However, the production method of the present invention is characterized in that by contrarily using as a source material such varieties of sugar cane of non-recommended having a higher fiber component content, almost of all the energy required in the production processes of the sugar and the useful material, especially the alcohol and/or the plastic, can be obtained from the fiber component. Further, using such varieties of sugar cane of higher dry matter yield amount as the source material makes it possible to increase the production amount of the sugar as well as that of the useful material, especially of the alcohol and/or the plastic, and accordingly the production method of the present invention can accomplish the improvement in productivity and energy-saving in producing the sugar and the useful material, especially the alcohol and/or the plastic, thus contributing to the development of the related industries. Conventionally, many of those varieties of sugar cane that have been recognized officially have typically a high content of cane sugar (sucrose) that is used as the source material for the sugar, as well as a low fiber component content in the stem portion aiming for improving the productivity. There are some varieties defined by high total yield amount, low cane sugar content and high fiber component content among those genetic resources that have been created through the breeding activities. Some of them have not yet considered as the officially recognized varieties from the reason as described above. If those unregistered varieties of sugar cane generic resource were used in the present system, owing to the large amount of the pressed residue of sugar cane to be generated, all of the energy required in the production process could be obtained by burning the pressed residue of sugar cane and also the insufficient cane sugar content could be compensated for by the increase in the total yield amount. Preferably, the sugar cane to be used in the present system is represented by one having the ratio of sugar to be produced (sucrose) of 7% by mass or greater in the cane stem region along with the total sugar of 10% by mass or greater.

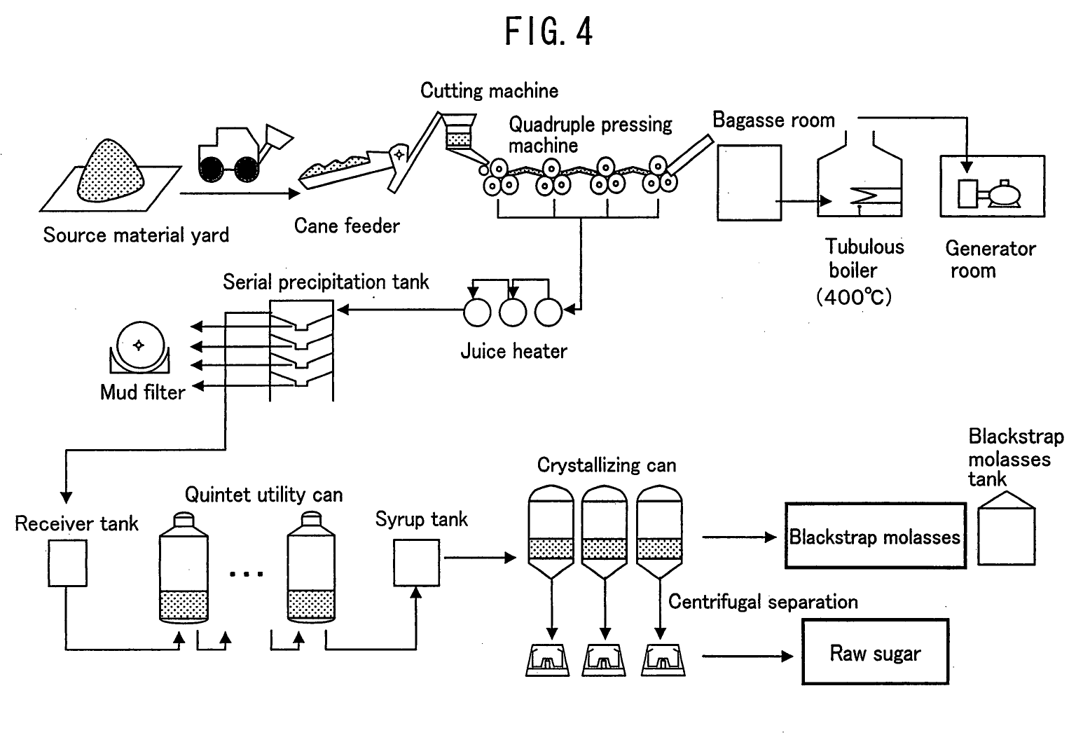

The process for producing the sugar and the blackstrap molasses from said pressed juice may be performed in accordance with any methods known to those skilled in the art, for example, by crystallizing the sugar. Specifically, said pressed juice is heated and concentrated by small portions (0.5-1 kl) under the vacuum by suction, which is repeated so as to take sugar crystal larger than a certain size. Then, a centrifugal separator is applied to separate the sugar crystal and the blackstrap molasses from each other. FIG. 4 shows an exemplary production flow in the production method of the raw sugar and the blackstrap molasses according to the present invention.

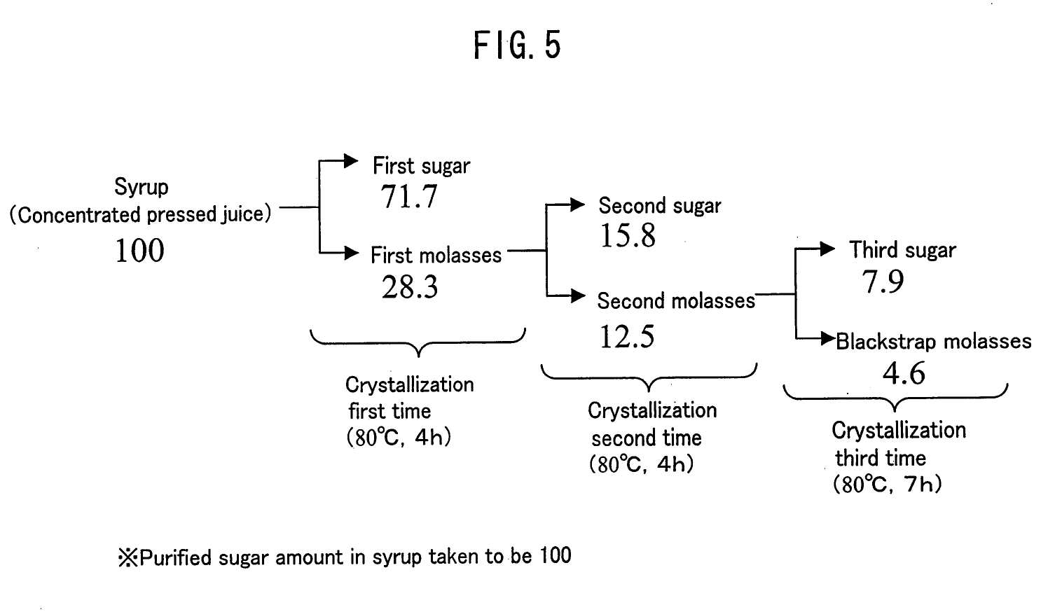

Preferably, said crystallizing process of the sugar may be performed by 2 times or less. As illustrated in FIG. 5, in the crystallization cycles of the sugar, the amount of sugar and thus the energy efficiency may be decreased over the increment of process cycle. In the present invention, using of the above specified sugar cane allows the efficient production of the ethanol even with the crystallizing process of sugar applied 2 times or less, yet advantageously without decreasing the amount of sugar to be produced. Further, the ethanol fermentation inhibitor, which is to be increased in proportion to the cycle of the crystallizing process, can be suppressed. In the present invention, preferably the crystallizing process of the sugar may be performed only once.

The process of generating the energy and the useful material by using the pressed juice, the blackstrap molasses and the pressed residue of sugar cane as source materials that have been obtained from said steps (a) and (b) may be carried our by any methods known to those skilled in the art. The useful material referred herein represents a fuel and materials made from sugar and vegetable cellulose taken as the base materials, including: for example, alcohol such as methanol, ethanol and butanol; flammable gas such as methane and hydrogen; biodegradable plastic made from sugar such as polylactic acid and polyhydroxy alkanoate taken as the base materials; and functional substance of microbial production such as amino acid and protein. In one embodiment of the present invention, the process for producing the ethanol from said blackstrap molasses may be carried out by any method known to those skilled in the art. As for the ethanol production method, such a method has been commonly practiced, in which the blackstrap molasses is processed by fermentable microorganism such as yeast so as to produce the ethanol. Besides, the method used for the fermentation may include a batch method in which the fermentable microorganism and the blackstrap molasses are blended in accordance with a specified ratio to take effect the fermentation and a serial method in which the fermentable microorganism is immobilized and then supplied with the blackstrap molasses continuously to take effect the fermentation. Further, as for the method for separating the produced ethanol by refining, a distillation method and a membrane separation method are known.

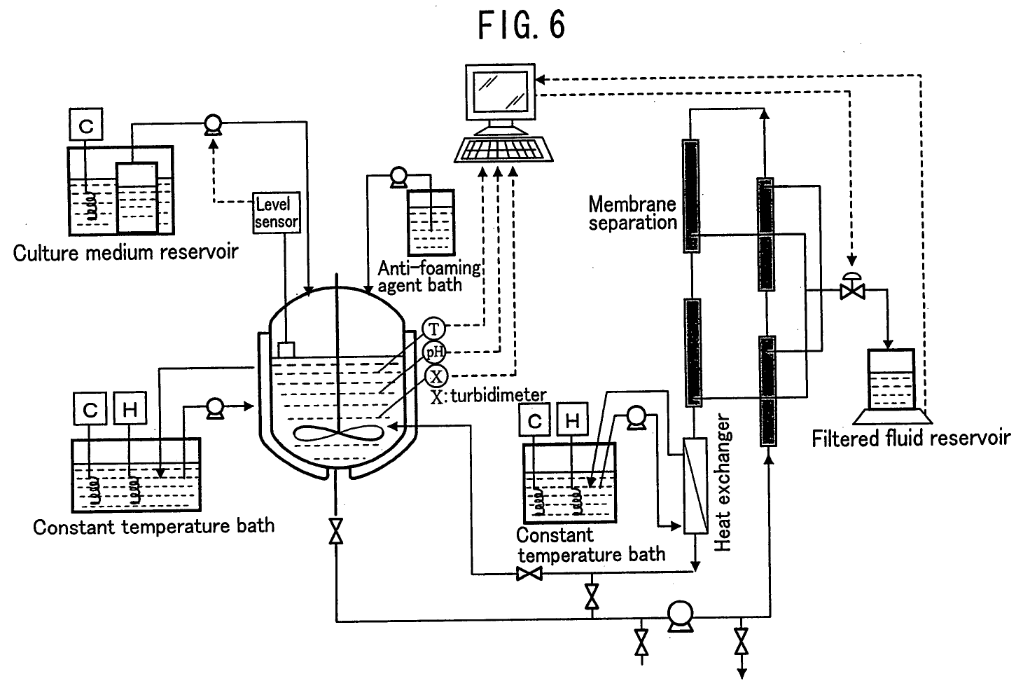

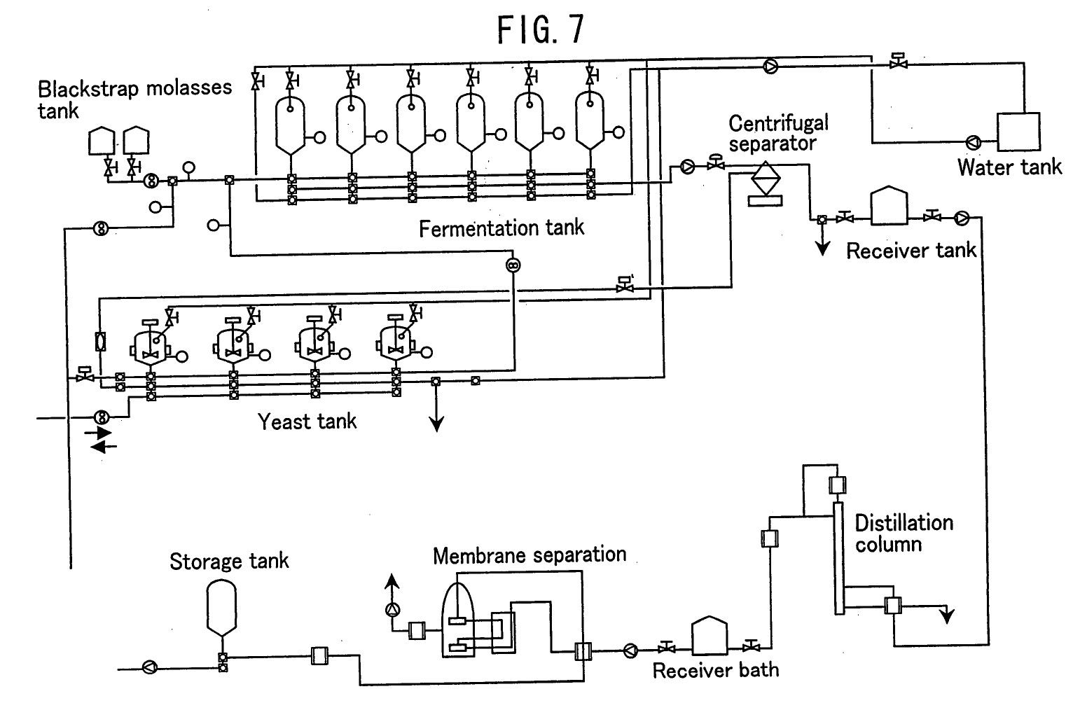

By way of example, the process may be carried out in accordance with the following method (see FIG. 6 and FIG. 7).

1) The fermentable microorganism: Japan Brewing Society's yeast, Society No. 7, for example, (the Saccharomyces cerevisiae).

2) The fermentation method: The yeast is immobilized in calcium alginate gel and the fermentation process is carried out at a temperature in a range of 10 to 20%. The produced ethanol is separated and refined through the distillation and the membrane separation process.

3) The culture solution: The blackstrap molasses is diluted adaptively to the sugar concentration of 20% for the application.

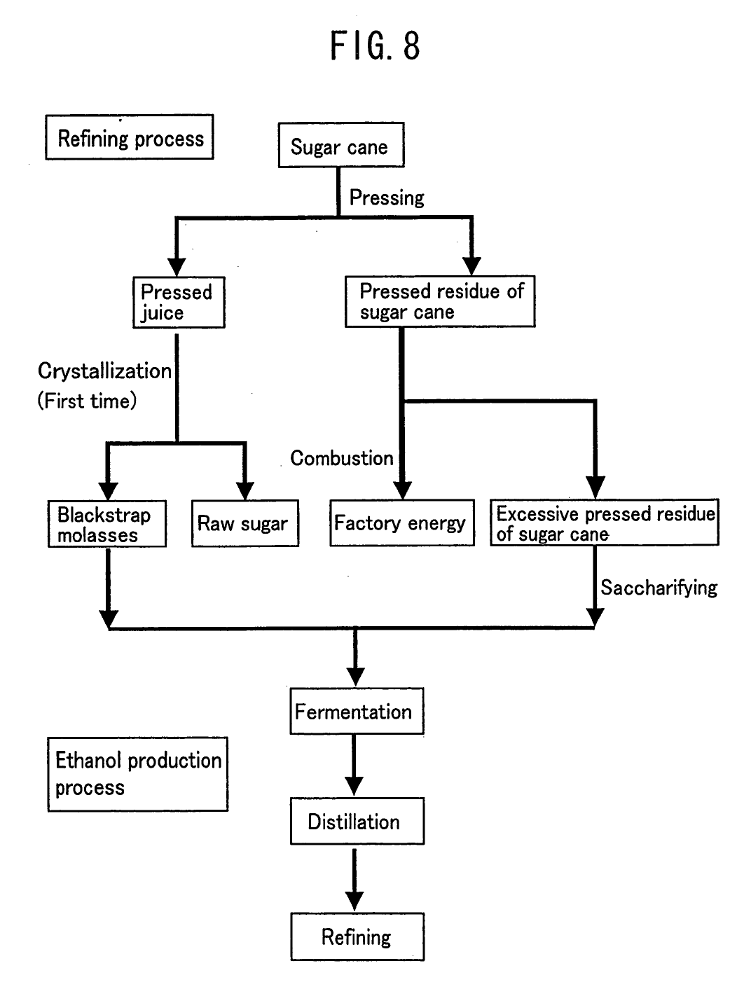

FIG. 8 schematically shows one example of the production method of the sugar and the ethanol according to the present invention.

It is to be noted that any excessive pressed residue of sugar cane that has been yielded excessively to the amount required for generating the energy for the process can be saccharified by using the method known to those skilled in the art so as be usable as a new source material for the fermentation.

The saccharifying process of the pressed residue of sugar cane may be carried out through, for example, the hydrolyzing by acid, the saccharifying by enzyme such as cellulase, and the hydrolyzing by water of high temperature and high pressure. Specifically, in the hydrolyzing by the acid, the pressed residue of sugar cane may be dipped in the acid, such as hydrochloric acid, sulfuric acid, to thereby cleavage a glucosidic linkage in the cellulose, which is a primary component of the pressed residue of sugar cane, and thus obtain glucose. The used acid may be recovered and reused. In the enzyme saccharifying by the cellulase, for example, the pressed residue of sugar cane may be crushed, undergo the pretreatment by the alkali treatment or the like, and then processed by the cellulase to thereby convert the cellulose, which is the primary component of the pressed residue of sugar cane, to the glucose. In the hydrolyzing by the water of high temperature and high pressure, for example, the pressed residue of sugar cane may be introduced into the water of high temperature and high pressure in a sub-critical or super-critical state at the temperature of 300% or higher to thereby decompose the cellulose, which is the primary component of the pressed residue of sugar cane, and thus obtain the glucose.

### EXAMPLE 1

### Production of Sugar and Ethanol

(Pressing Process)

Cane stem portions of the reaped sugar cane (97S-133) are cut by a cutter (13 to 72 pieces of knives, 375-675 rpm) into 15-30 cm long pieces and then finely shredded by a shredder. The shredded sugar cane is pressed by a mill roll comprising sets of three rolls arranged in the quadruple (12 rolls) or quintet (15 rolls) configuration so as to press the saccharic juice out of the sugar cane. In order to improve the pressing-out rate, the last set of rolls may be supplied with the water to allow 95 to 97% of saccharic component to be pressed out. The sugar concentration of the pressed juice is in a range of Bx13 to 15. Subsequently, the saccharic juice is heated up to 80-100° C. by the juice heater (effective heating area of 4 m²) and placed in a lime mixing bath, where ash (pH 7.6-8.0, 0.07% CaO (relative to the sugar cane)) is added to the saccharic juice so as to precipitate any impurities (supernatant fluid is supplied to the concentrating process) and then filtered by the Oliver filter (revolving speed of 6 rpm, cake amount of 2-4% (relative to the sugar cane), washing volume: 150% of the cake, saccharic component of the cake: 0.8-1.7%), and the filtered fluid is sent to the concentrating process. The supernatant fluid and the filtered fluid are continuously concentrated by evaporation under a vacuum condition in a quadruple utility can to thereby obtain the pressed juice (Bx60).

(Crystallizing Process)

In a sugar crystallizer can, every small portion (0.5-1 kl) of the pressed juice obtained in the concentrating process is heated and concentrated under the vacuum by suction, which is repeated so as to take out sugar crystal of a certain size (Bx92-93). Subsequently, a centrifugal separator is used to separate every certain amount thereof (200-400 liters) into the sugar crystal and the blackstrap molasses (1200-1500 rpm, cycle by 5-10 minutes, lower net of 8 mesh, upper net of 0.35).

(Ethanol Production Process)

The pure separated yeast strain (Japan Brewing Society's No. 9 yeast) was planted in a test tube containing a culture medium for an advanced culture 1 (glucose 2.0% (w/v), Yeast Nitrogen Base (w/o:AA-AS) 0.17% (w/v), ammonia sulfate 0.5% (w/v)) and then underwent the shake culture at 30° C. for 12 hours (125 rpm). Subsequently, the yeast was planted in a Sakaguchi flask (quantity of 500 ml) containing a culture medium for an advanced culture 2 (glucose 2.0% (w/v), Yeast Extract 1.0% (w/v), Bacto Peptone 2.0% (w/v)) to yield 2×10⁶ cell/ml and then underwent the shake culture at 30° C. for 6 hours (125 rpm), thereby having collected the yeast in the logarithmic growth period (after the fourth generation in growth) for the fermentation.

Thus obtained yeast was planted to yield 2×10⁷ cell/ml and then transferred to a culture medium for fermentation of 500 ml in an Erlenmeyer flask, where it was fermented at 30° C. for the ethanol. The blackstrap molasses separated in the crystallizing process was prepared so as to yield the saccharic concentration of 10% (w/v), which was in turn to be used as a culture medium for fermentation. It was left under the anaerobic conditions for 3 days for the fermentation. After the completion of the fermentation, the fermented liquid was filtered by a membrane filter having a perforation diameter of 0.45 μm, and then the ethanol concentration was measured in accordance with the gas chromatography. Obtained was the fermented liquid of ethanol of 4.5% (w/v).

### EXAMPLE 2

### Produced Amount and Energy Calculation Obtainable from High-Yielding sugar cane 95GA-27 and from conventional variety (sugar cane of common variety)

The production amount of the raw sugar and the ethanol as well as the generated amount of the energy obtainable from the high-yielding sugar cane 95GA-27 and from the conventional variety (sugar cane of common variety) were calculated for different number of cycle of the crystallizing process. The examples 1 through 3 represent a case where an entire amount of the obtainable bagasse was burnt, the examples 4 through 6 represent a case where a certain amount of bagasse for the required energy was burnt and the remaining amount of bagasse, after the saccharifying process, was used for the ethanol production, and the comparative examples 1 through 3 represent a case where an entire amount of obtainable bagasse was burnt. Table 4 shows the calculation results.

It is to be noted that respective values were calculated in the following manner.

(1) Raw Sugar, Ethanol and Bagasse Production Amounts

The data indicated in Table 2 were used to calculate the raw sugar, the ethanol and the bagasse production amounts, respectively, by using the following equations.
1. Raw sugar production amount [t/ha]=Sugar cane unit yield amount [t/ha]×Ratio of sugar to be produced [%]/100×Pressing efficiency [%]/100×(100−Purification loss) [%]/100×Crystallization yield ratio [%]/100×(100−Centrifugal loss) [%]/100;
2. Amount of sugar to be produced in blackstrap molasses [t/ha]
=Sugar cane unit yield amount [t/ha]×Ratio of sugar to be produced [%]/100×Pressing efficiency [%]/100×(100-Purification loss) [%]/100×(100-Crystallization yield ratio) [%]/100×(100-Centrifugal loss) [%]/100;
3. Amount of sugar not to be produced in blackstrap molasses
=Sugar cane unit yield amount [t/ha]×Ratio of sugar not to be produced [%]/100×Pressing efficiency [%]/100×(100-Purification loss) [%]/100×(100-Centrifugal loss) [%]/100;
4. Ethanol production amount[kL/ha]
=(Amount of sugar to be produced in blackstrap molasses [t/ha]×0.69 [kL/t]+Amount of sugar not to be produced in blackstrap molasses [t/ha]×0.655 [kL/t])×Fermention efficiency [%]/100
5. Bagasse production amount [t/ha]
=Sugar cane unit yield amount [t/ha]×Fiber component content [%]/100×100/(100-Moisture content)[%]
Sugar to be produced (sucrose)
   C₁₂H₂₂O₁₁+H₂O→4C₂H₅OH+4CO₂
1 mol (342 g) 4 mol (184 g)
Sugar not to be produced (glucose, fructose)
   C₆H₁₂O₆→2C₂H₅OH+2CO₂
1 mol (180 g) 2 mol (92 g)
Theoretical yield amount
   Sugar to be produced 1 [g]→Ethanol 0.538 [g]=0.690 [ml]

Sugar not to be produced 1 [g]→Ethanol 0.511 [g]=0.655 [ml]

TABLE 2
Data used for calculation

| Pressing efficiency | | 95% |
|---|---|---|
| Purification loss | | 1.5% |
| Crystallization yield ratio | One time | 71.7% |
| | Two times | 87.5% |
| | Three times | 95.4% |
| Centrifugal loss | Sugar to be produced | 5% |
| | Sugar not to be produced | 10% |

| | |
|---|---|
| Fermentation efficiency | 95% |
| Bagasse moisture content | 50% |

(2) Combustion Energy of Bagasse

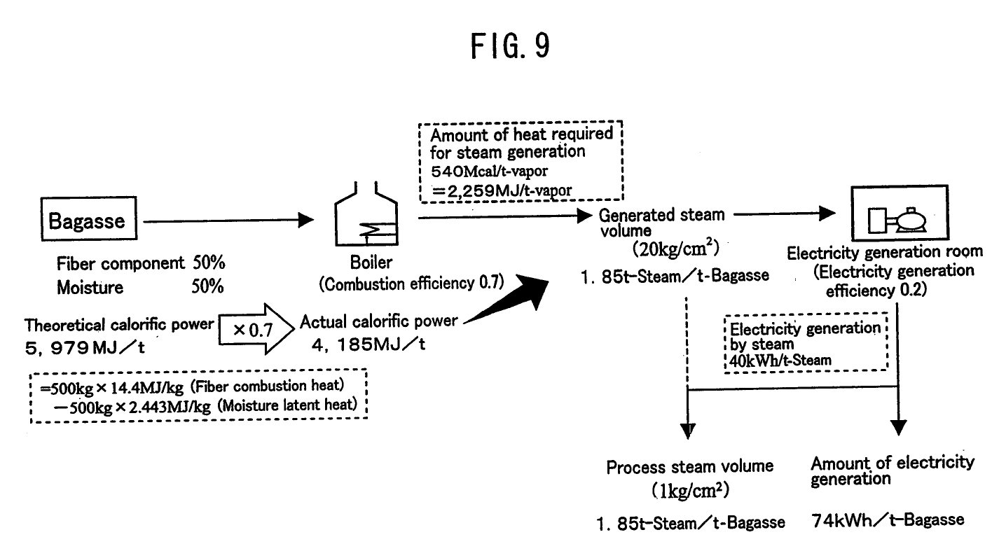

The combustion energy of bagasse was calculated in accordance with the theoretical consideration illustrated in FIG. 9. The obtained combustion energy of bagasse was 1.85 ton per one ton of bagasse in the representation by the steam volume, and 74 kWh per one ton of bagasse in the representation by the electricity generation.

(3) Energy Necessary for Producing Raw Sugar

The steam volume required for producing raw sugar was determined as the steam volume per one ton of source material based on the consideration of the combustion energy of bagasse described above from the viewpoint of the fuel consumption for bagasse and heavy oil in the table on page 80 in “Heisei 13/14, Sugar production record by sugar cane and sweet potato” (Agriculture, Forestry and Fisheries Section, Okinawa Prefecture). Besides, the electricity generation required for the production of the raw sugar was determined based on the data provided on page 43 of “Raw sugar production method” (by Takeo Yamane, issued by Sugar Production Technology Study Group). Further, the steam volume and the electricity generation for the cycle of the crystallizing process that was reduced to once and twice were calculated based on the data provided in Table 2-1 and Table 2-3 on page 41-43 in “Raw sugar production method” (by Takeo Yamane, issued by Sugar Production Technology Study Group). That is, the energy corresponding to each part of “decocting of sugar, stimulating crystallization, and curing of sugar” involved in the crystallizing process was divided by three and allocated depending on the number of cycles for the calculation. Table 3 shows the obtained steam volume and the electricity generation required for the raw sugar production.

TABLE 3
Energy required for raw sugar production

| | Required steam | Required electric |
|---|---|---|
| | volume [t-steam/ | power [kWh/t- |
| | t-cane sugar] | cane sugar] |
| 3-time crystallization | 0.470 | 18.0 |
| 2-time crystallization | 0.418 | 16.7 |
| 1-time crystallization | 0.366 | 15.4 |

(4) Energy Required for Ethanol Production

The steam volume and the electricity generation required for the ethanol production was determined from an average over the production data B, C and D indicated in Table 11 on page 262 in “By-product in sugar production industry—Introduction to industrial use—” (Japan Blackstrap Molasses Industry Association). The obtained energy is 5.38 ton per 1 kL of ethanol in the representation by the steam volume and 120 kWh per 1 kL of ethanol in the representation by the electricity generation.

| | | | Example 1 | Example 2 | Example 3 | Example 4 | Example 5 | Example 6 |
|---|---|---|---|---|---|---|---|---|
| | Sugar cane variety | | 95GA27 | | | 95GA27 | | |
| | Crystallization cycle | | 3-time | 2-time | 1-time | 3-time | 2-time | 1-time |
| | Raw sugar crystal- | [%] | 95.4 | 87.5 | 71.7 | 95.4 | 87.5 | 71.7 |
| | lization yield | | | | | | | |
| | ratio | | | | | | | |
| Variety property | Unit yield amount | [t/ha] | 162.5 | | | 162.5 | | |
| of sugar cane | Dry matter yield | [dry-t/ha] | 66.4 | | | 66.4 | | |
| variety | amount | | | | | | | |
| | Ratio of sugar to be | [%] | 8.8 | | | 8.8 | | |
| | produced | | | | | | | |
| | Ratio of sugar not to | [%] | 3.6 | | | 3.6 | | |
| | be produced | | | | | | | |
| | Fiber content | [%] | 20.5 | | | 20.5 | | |
| Final production | Raw sugar production | [t/ha] | 12.13 | 11.12 | 9.11 | 12.13 | 11.12 | 9.11 |
| amount | amount | | | | | | | |
| | Ethanol production | [kL/ha] | 3.47 | 4.16 | 5.55 | 5.05 | 6.00 | 7.44 |
| | amount | | | | | | | |
| Supply of | Bagasse production | [t/ha] | 66.63 | | | 55.96 | 54.18 | 53.80 |
| bagasse | amount (combustion | | | | | | | |
| combustion | volume) | | | | | | | |
| energy | Generated steam | [t/ha] | 123.26 | | | 103.53 | 100.23 | 99.53 |
| | volume from bagasse | | | | | | | |
| | combustion | | | | | | | |
| | Electricity generation | [kWh/ha] | 4930 | | | 4141 | 4009 | 3981 |
| | from bagasse | | | | | | | |
| | combustion | | | | | | | |
| Steam | Producing raw sugar | [t/ha] | 76.38 | 67.93 | 59.48 | 76.38 | 67.93 | 59.48 |
| consumption | Producing ethanol | [t/ha] | 18.66 | 22.39 | 29.85 | 27.14 | 32.29 | 40.04 |
| Electric power | Producing raw sugar | [kWh/ha] | 2925 | 2714 | 2503 | 2925 | 2714 | 2503 |
| consumption | Producing ethanol | [kWh/ha] | 416 | 499 | 666 | 605 | 720 | 893 |
| Bagasse | Ratio of bagasse | [%] | 130 | 136 | 138 | 100 | 100 | 100 |
| combustion | steam contribution to | | | | | | | |
| energy | total energy | | | | | | | |
| contribution | consumption | | | | | | | |
| | Ratio of bagasse | [%] | 148 | 153 | 156 | 117 | 117 | 117 |
| | generated electric | | | | | | | |
| | power to total energy | | | | | | | |
| | consumption | | | | | | | |

| | | | Comparative | Comparative | Comparative |
|---|---|---|---|---|---|
| | | | example 1 | example 2 | example 3 |
| | Sugar cane variety | | Conventional variety (Average value) | | |
| | Crystallization cycle | | 3-time | 2-time | 1-time |
| | Raw sugar crystal- | 95.4 | 95.4 | 95.4 | 71.7 |
| | lization yield | | | | |
| | ratio | | | | |
| Variety property | Unit yield amount | [t/ha] | 64.7 | | |
| of sugar cane | Dry matter yield | [dry-t/ha] | 26.4*¹ | | |
| variety | amount | | | | |
| | Ratio of sugar to be | [%] | 13.3 | | |
| | produced | | | | |
| | Ratio of sugar not to | [%] | 3.6*² | | |
| | be produced | | | | |
| | Fiber content | [%] | 13.3 | | |
| Final production | Raw sugar production | [t/ha] | 7.30 | 7.30 | 7.30 |
| amount | amount | | | | |
| | Ethanol production | [kL/ha] | 1.46 | 1.46 | 1.46 |
| | amount | | | | |
| Supply of | Bagasse production | [t/ha] | 17.21 | | |
| bagasse | amount (combustion | | | | |
| combustion | volume) | | | | |
| energy | Generated steam | [t/ha] | 31.84 | | |
| | volume from bagasse | | | | |
| | combustion | | | | |
| | Electricity generation | [kWh/ha] | 1274 | | |
| | from bagasse | | | | |
| | combustion | | | | |
| Steam | Producing raw sugar | [t/ha] | 30.41 | 27.04 | 23.68 |
| consumption | Producing ethanol | [t/ha] | 7.87 | 10.12 | 14.60 |
| Electric power | Producing raw sugar | [kWh/ha] | 1165 | 1080 | 996 |
| consumption | Producing ethanol | [kWh/ha] | 176 | 226 | 326 |
| Bagasse | Ratio of bagasse | [%] | 83 | 86 | 83 |
| combustion | steam contribution to | | | | |
| energy | total energy | | | | |
| contribution | consumption | | | | |
| | Ratio of bagasse | [%] | 95 | 98 | 96 |
| | generated electric | | | | |
| | power to total energy | | | | |
| | consumption | | | | |

*¹Calculated by applying the same ratio of dry matter yield amount/unit yield amount of 95GA-27.
*²Considered to be equivalent to 95GA-27

As obvious from Table 4, by using the method of the present invention, the ethanol production amount could be greatly increased as compared to the prior art method, and also by using the method of the present invention, gasse, while in the conventional method, it 10 was impossible to obtain all the energy required for the raw sugar production and the ethanol production from the combustion energy of the bagasse.

### EXAMPLE 3

### Production of Raw Sugar and Blackstrap Molasses Using High Yielding Sugar Cane, 95GA-27 (Laboratory Scale)

(1) Pressing of Sugar Cane/Clarification of Pressed Juice

Cane stem portions weighing about 3 kg of reaped sugar cane (95GA-27) were cut by a shredder and then pressed by a quadruple mill roll unit, thereby having obtained pressed juice of 2 L (sugar concentration Bx=15.2). The pressed juice was transferred into a 3 L Erlenmeyer flask and heated up to 70° C. in a water bath, and then further added with 1.00 g (0.05% relative to the pressed juice weight) of Ca(OH)₂ and stirred for 30 minutes to thereby precipitate impurities contained therein. Subsequently, the resultant composition was centrifugally separated by an angle rotor type centrifugal separator at 800 rpm for 10 minutes to thereby separate the supernatant clarified pressed juice and the sediment from each other.

(2) Concentrating and Crystallizing of Clarified Pressed Juice

The clarified pressed juice obtained in the above process was concentrated in a rotary evaporator having a capacity of 3 L at a temperature within the flask of 50° C. under vacuum by suction (70-110 mmHg) for 4 hours (evaporated moisture content of 1700 mL), thereby having obtained about 300 mL of concentrated syrup (Bx=80.0).

(3) Crystallizing of Concentrated Syrup

The concentrated syrup was added with 50 g of commercially available granulated sugar (granular size in a range of 250-500 μm) as a seed crystal and crystallized at a temperature within the flask of 50° C. under vacuum by suction (120 mmHg) for 4 hours.

(4) Separation of Raw Sugar and Molasses from Each Other

The mixture of sugar and molasses obtained in the above process was centrifugally separated in a perforated wall type centrifugal separator using a filter cloth of 50-100 μm mesh at 3000 rpm for 20 minutes, and thus separated into crystallized sugar (a first sugar) and molasses (first molasses). The recovered first sugar was dried and cooled over a night and weighed so as to determine a yield amount by subtracting an added amount of seed crystal.

(5) Re-Crystallization of Molasses

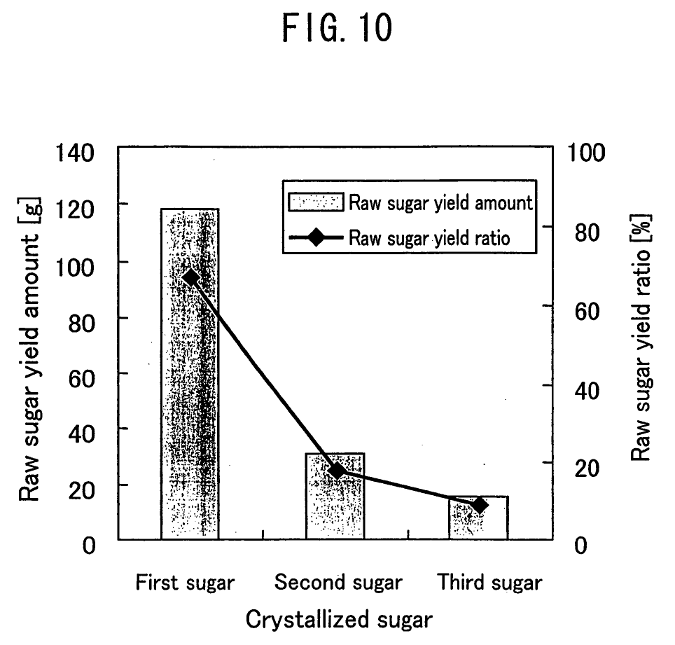

The molasses obtained in the above process (the first molasses) had a water poured to meet the Bx=80, and then the procedures in the processes (3) and (4) were repeated to thereby obtain a second sugar and a second molasses. After another pouring of the water, the procedures in the processes (3) and (4) were repeated again to thereby obtain a third sugar and a third molasses (blackstrap molasses). FIG. 10 illustrates a relationship between the number of cycles of the crystallizing process and the raw sugar yield ratio.

As obvious from FIG. 10, the raw sugar yield ratio is about 70% for the first sugar and about 90% for the first sugar added with the second sugar entirely.

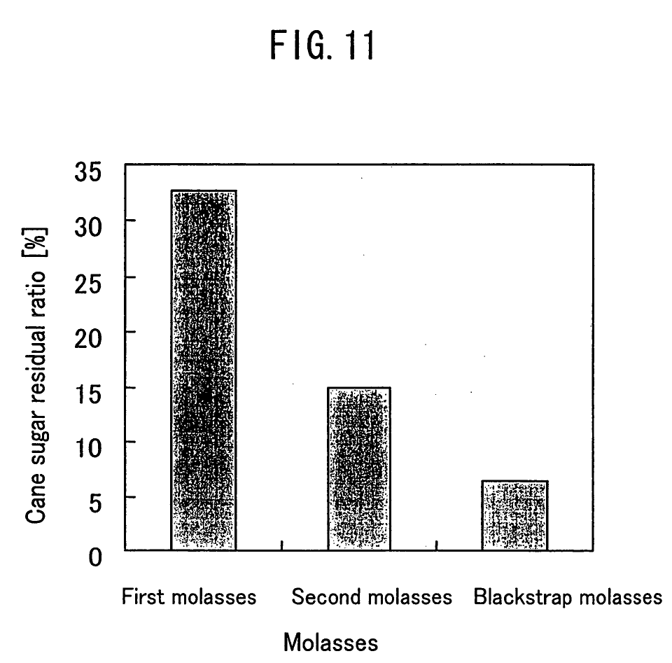

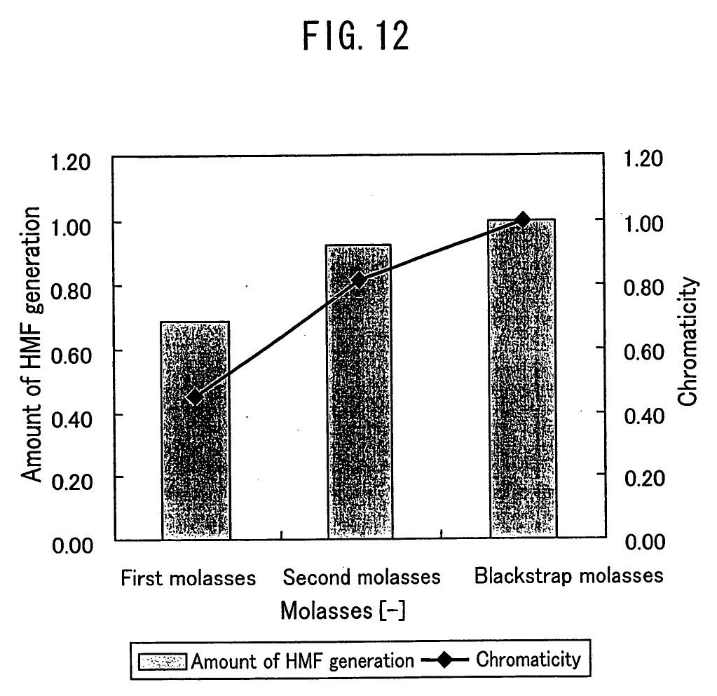

For the molasses obtained in the above process (the first molasses and the second molasses), the cane sugar residual ratio, the generated amount of HMF (hydroxymethyl furfural) representing a fermentation inhibitor and a chromaticity were measured and compared with the corresponding values of the blackstrap molasses (the third syrup) obtained by the conventional method. The cane sugar residual ratio was calculated by subtracting the yield ratios of the respective crystallized sugars based on the assumption that the cane sugar volume contained in the concentrated syrup of the example 1 is 100%. As for the HMF, determination was made in accordance with the method described on page 682 in the “Sugar Handbook” (edited by Eijiro Hamaguchi and Yoshito Sakurai, Asakura book company, 1964) (i.e., the method in which a difference between an absorbance of the wavelength of 284 μm and an absorbance of the wavelength of 245 μm is determined from an analytical curve for a known concentration). After the object was diluted by the water to be 30 times and put into a quartz cell, the chromaticity was determined by a colorimeter (EBC). FIG. 11 and FIG. 12 show the result.

It is seen from FIG. 11 that the cane sugar residual ratio is higher with a lower number of cycles of the crystallizing process, wherein if those are used as the source material for the ethanol fermentation, the ethanol yield amount will be increased.

Besides, it is also seen from FIG. 12 that the generated amount of HMF, which is a fermentation inhibitor, and the chromaticity are decreased for the lower number of cycles of the crystallizing process. That is, using the molasses that has undergone lesser times of crystallizing process can exhibit a better fermentation and also reduce the problem of coloring of the drain water.

What is claimed is:

1. A method for producing sugar and a useful material from a sugar cane, comprising the steps of:
(a) producing from sugar cane a pressed juice and a pressed residue of sugar cane;
(b) producing sugar and blackstrap molasses from said pressed juice; and
(c) generating an energy and a useful material by using said pressed juice, said blackstrap molasses and said pressed residue of sugar cane as source materials that have been obtained from said steps (a) and (b), wherein
said sugar cane contains an amount of 15% or greater by mass of fiber components in its cane stem region and provides a dry matter yield amount per unit area of 40 t/ha/year or higher; and
90% or more of energy required for all of the steps of said production method is obtained from energy generated by burning said pressed residue of sugar cane.

2. A method of claim 1, wherein said useful material is selected from the group consisting of alcohol, flammable gas, biodegradable plastic made from sugar taken as the base materials, and functional substance of microbial production.

3. A method of claim 1, wherein said useful material is alcohol or biodegradable plastic made from sugar taken as the base materials.

4. A method of claim 1, wherein said sugar cane contains an amount of 15 to 25% by mass of fiber components in its cane stem region.

5. A method of claim 1, wherein said dry matter yield amount per unit area is 65 t/ha/year or higher.

6. A method of claim 1, wherein said dry matter yield amount per unit area is 80 t/ha/year or higher.

7. A method of claim 1, wherein said step (c) comprises a process of producing ethanol from said blackstrap molasses that has been obtained from said step (b).

8. A method of claim 7, wherein said step (b) comprises two or less times of crystallizing process of sugar.

9. A method of claim 8, wherein said step (b) comprises one time of crystallizing process of sugar.

10. A method of claim 7, wherein 95% or more of energy required to be consumed in all of the steps of said production method is obtained from energy generated by burning said pressed residue of sugar cane.
